(19) United States
(12) Patent Application Publication
(10) Pub. No.: US 2023/0355160 A1
(43) Pub. Date: Nov. 9, 2023

(54) WEARABLE SURFACE ELECTROMYOGRAPHY SENSOR UNITS AND METHODS OF USE AND FABRICATION

(71) Applicant: Purdue Research Foundation, West Lafayette, IN (US)

(72) Inventors: Georgia Malandraki, West Lafayette, IN (US); Kevin L. Berry, Lafayette, IN (US)

(21) Appl. No.: 18/308,816

(22) Filed: Apr. 28, 2023

Related U.S. Application Data
(60) Provisional application No. 63/338,112, filed on May 4, 2022.

(51) Int. Cl. A61B 5/389; A61B 5/00; A61B 5/296

(52) U.S. Cl. CPC A61B 5/389; A61B 5/6832; A61B 5/6814; A61B 5/486; A61B 5/296; A61B 2562/164

(57) ABSTRACT

A wearable surface electromyography (sEMG) sensor unit and associated methods. The sensor unit has a flexible non-stretchable substrate comprising a first layer defining a first side of the substrate and a second layer defining a second side of the substrate, one or more sensor electrodes including at least a first pair of active electrodes and a ground electrode disposed on the first side, a plurality of traces disposed on the second side, and vias connecting the traces and sensor electrodes. The substrate may include one or more reinforcing layers at areas of high stress to increase durability of the sensor unit.

### CROSS-REFERENCE TO RELATED APPLICATIONS

This application claims the benefit of U.S. Provisional Application No. 63/338,112, filed May 4, 2022, the contents of which are incorporated herein by reference.

### STATEMENT REGARDING FEDERALLY SPONSORED RESEARCH

This invention was made with government support under Grant 1R21EB026099-01A1 awarded by National Institutes of Health. The government has certain rights in the invention.

### BACKGROUND OF THE INVENTION

The invention generally relates to wearable surface electromyography (hereinafter, also referred to as “sEMG”) sensors and methods of making and/or using the same. More specifically, some nonlimiting aspects of the invention relate to devices and methods for the optimization of a wearable sEMG sensor for the rehabilitation of oropharyngeal dysphagia.

Swallowing disorders (a.k.a. dysphagia) are known to affect more than 10 million adults and over 0.5 million children in the US per year. With COVID-19 this number is expected to increase because dysphagia is common among COVID-19 survivors. Unfortunately, the inability to swallow can have detrimental consequences on physical and emotional health, quality of life, and healthcare costs. Therefore, effective management of dysphagia is desired in order to prevent these life-altering ramifications. Currently, the most common rehabilitative swallowing treatments target the strength and/or coordination of oropharyngeal muscles.

The use of biofeedback as an adjunct to these treatments has been shown to improve specific swallow physiology events mostly in small-scale studies and is gradually gaining popularity in clinical practice. However, most currently available biofeedback devices for dysphagia management, such as surface electromyography (sEMG), manometry, and endoscopy, are large, and/or expensive, and thus primarily available in large urban clinical centers and not easily transferrable to the home setting or adaptable to use for telehealth. However, telehealth for dysphagia management has seen a significant expansion in the COVID-19 era, which has further highlighted the need for reliable, user-friendly, and affordable telehealth systems for swallowing management.

In recent years, the development of portable, cloud-based, or wearable devices for rehabilitation of dysphagia has emerged. In the area of surface EMG specifically, new technologies such as rigid wearable sensor designs that accommodate signal acquisition from one side of the neck, or bilateral ultrathin flexible wearable sensors (e.g., Kim et al., “Flexible submental sensor patch with remote monitoring controls for management of oropharyngeal swallowing disorders,” Sci Adv. 2019 Dec. 13;5(12):eaay3210, which is incorporated herein by reference in its entirety) have been developed. However, these promising technologies either remain relatively expensive, or have a short lifetime and low durability for clinical use.

For example, an ultrathin flexible and stretchable sEMG sensor patch has been developed for the submental area that uses a honeycomb design, and its use has been validated against commercially available snap-on sEMG sensors, as disclosed in Kantarcigil et al., “Validation of a Novel Wearable Electromyography Patch for Monitoring Submental Muscle Activity During Swallowing: A Randomized Crossover Trial,” J. Speech Lang. Hear. Res., 2020 Oct. 16;63(10):3293-3310 (hereinafter, Kantarcigil), which is incorporated herein by reference in its entirety. Though the technical performance of this patch was comparable to existing commercial sEMG sensors, in pre-clinical trials it was recognized that durability, complexity, and ease of application were aspects in which improvements would be desirable.

Therefore, there is an ongoing desire for wearable sEMG sensors adapted to collect surface EMG signals from the head and neck area of an individual, and are capable of maintaining high technical performance and durability after repeated uses.

### BRIEF SUMMARY OF THE INVENTION

The intent of this section of the specification is to briefly indicate the nature and substance of the invention, as opposed to an exhaustive statement of all subject matter and aspects of the invention. Therefore, while this section identifies subject matter recited in the claims, additional subject matter and aspects relating to the invention are set forth in other sections of the specification, particularly the detailed description, as well as any drawings.

The present invention provides, but is not limited to, wearable surface electromyography (sEMG) sensor units and methods of making and using the same.

According to a nonlimiting aspect of the invention, a wearable surface electromyography (sEMG) sensor unit includes a flexible non-stretchable substrate comprising a first layer defining a first side of the substrate and a second layer defining a second side of the substrate. A plurality of sensor electrodes, including at least one pair of active electrodes and a ground electrode, are disposed on the first side. The plurality of sensor electrodes. Traces are disposed on the second side and one or more vias connect the traces with the sensor electrodes.

According to another nonlimiting aspect of the invention, a method is provided for obtaining sEMG biofeedback for the treatment of a patient with dysphagia. The method includes placing a wearable sEMG sensor unit in a use position on the submental area of the patient such that an active electrode is located near a submental muscle of interest, and obtaining signals from the active electrode indicative of activity of the submental muscle of interest.

According to yet another nonlimiting aspect of the invention, a method of fabricating a wearable sEMG sensor unit includes forming a flexible non-stretchable substrate to comprise a polymeric layer and a metallic layer, forming sensor electrodes on a first side of the substrate, forming traces on a second side of the substrate, and connecting the sensor electrodes and traces with vias.

Technical aspects of sensor units and methods as described above preferably include the capability of exhibiting good adhesion, high technical performance, durability, and/or reliable signal quality throughout multiple uses.

Other aspects and advantages of this invention will be appreciated from the following detailed description.

### BRIEF DESCRIPTION OF THE DRAWINGS

FIG. 1A is a plan view schematically representing a front side of an sEMG sensor unit according to a nonlimiting embodiment of the invention.

FIG. 1B is a plan view schematically representing a back side of the sEMG sensor unit of FIG. 1A.

FIG. 2 shows the sEMG sensor unit of FIGS. 1A and 1B applied to a submental area of a person according to a nonlimiting aspect of the invention.

FIGS. 3A and 3B schematically represent back and front sides, respectively, of an sEMG sensor unit according to another nonlimiting embodiment of the invention.

FIG. 4 illustrates signal to noise ratios across sessions for two different individuals across multiple uses.

### DETAILED DESCRIPTION OF THE INVENTION

The intended purpose of the following detailed description of the invention and the phraseology and terminology employed therein is to describe what is shown in the drawings, which relate to one or more nonlimiting embodiments of the invention, and to describe certain but not all aspects of what is depicted in the drawings, including the embodiment(s) depicted in the drawings. The following detailed description also describes certain investigations relating to the embodiment(s) depicted in the drawings, and identifies certain but not all alternatives of the embodiment(s) depicted in the drawings. As nonlimiting examples, the invention encompasses additional or alternative embodiments in which one or more features or aspects shown and/or described as part of a particular depicted embodiment could be eliminated, and also encompasses additional or alternative embodiments that combine two or more features or aspects shown and/or described as part of different embodiments. Therefore, the appended claims, and not the detailed description, are intended to recite particularly point out subject matter regarded to be aspects of the invention, including certain but not necessarily all of the aspects and alternatives described in the detailed description.

Although the invention will be described below in reference to wearable sEMG sensor units shown in the drawings, it will be appreciated that the teachings of the invention are more generally applicable to a variety of types of wearable sensors. To facilitate the description provided below of the embodiment(s) represented in the drawings, relative terms, including but not limited to, “proximal,” “distal,” “anterior,” “posterior,” “vertical,” “horizontal,” “lateral,” “front,” “rear,” “side,” “forward,” “rearward,” “top,” “bottom,” “upper,” “lower,” “above,” “below,” “right,” “left,” etc., may be used in reference to the orientation of a wearable sEMG sensor unit during its use and/or as represented in the drawings. All such relative terms are useful to describe the illustrated embodiment(s) but should not be otherwise interpreted as limiting the scope of the invention.

The present invention provides wearable sEMG sensor units. The units are preferably capable of use as a component of a biofeedback device and being configured to address one or more limitations of other technologies as outlined previously herein. The wearable sEMG sensor units are preferably thin (as opposed to ultrathin) and fabricated on a substrate that is relatively flexible (capable of flexing out of the plane of the sensor) and yet relatively non-stretchable (limited strain in the plane of the sensor).

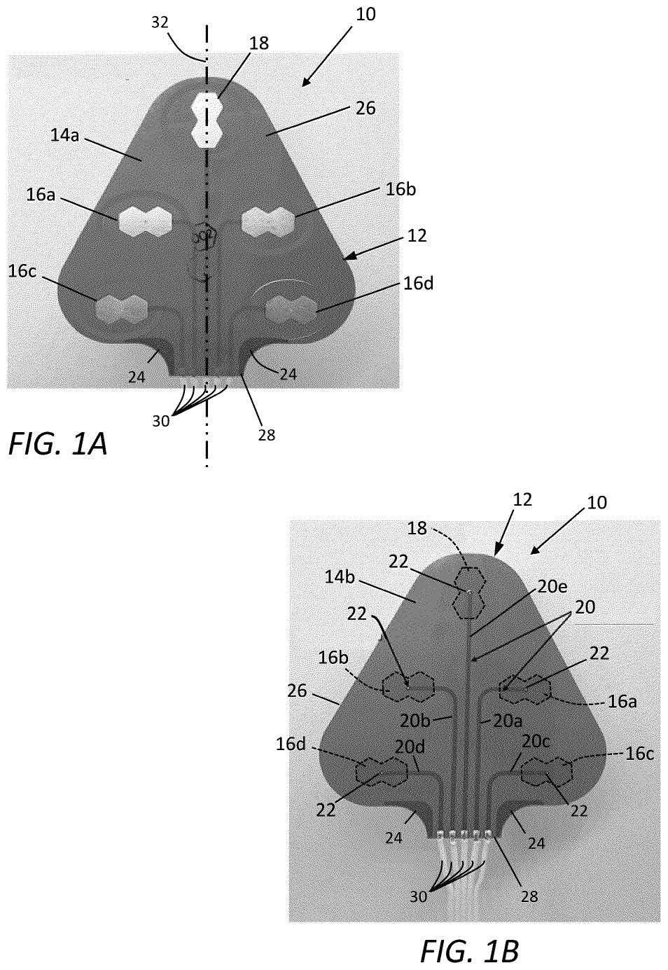

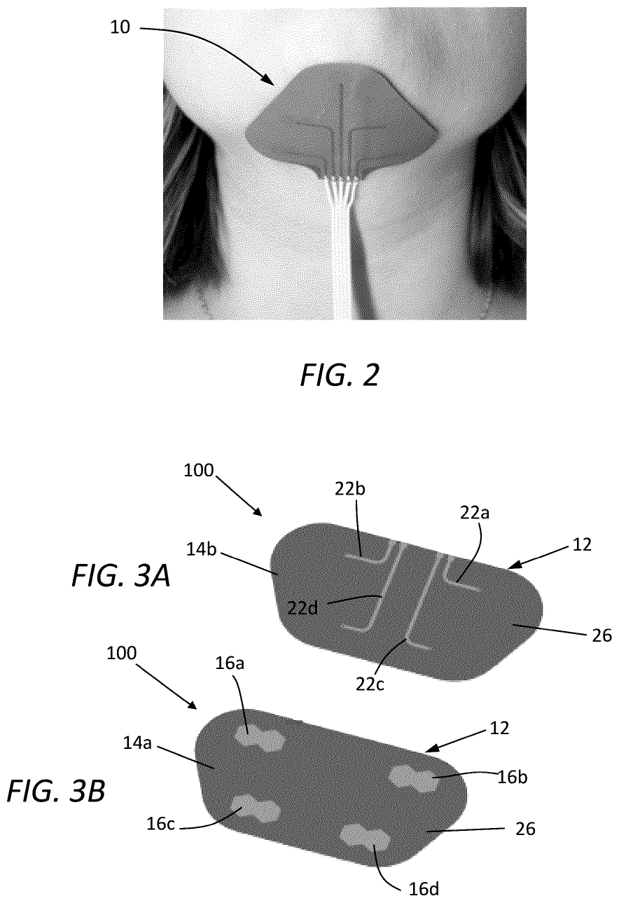

Turning now to the nonlimiting embodiments represented in the drawings, FIGS. 1A, 1B, and 2 depict a nonlimiting embodiment of a wearable surface electromyography (hereinafter, “sEMG” or “surface EMG”) sensor unit 10 (which may also be referred to as an “sEMG patch,” a “sensor patch,” or similar) in accordance with certain nonlimiting aspects of the invention. The sensor unit 10 is shown in the form of a thin, flexible, non-stretchable sEMG patch with two sides bearing different components of the unit 10.

As best seen in FIGS. 1A and 1B, the sensor unit 10 comprises a substrate 12 that is preferably thin, flexible, and non-stretchable. The substrate 12 has opposite front and back sides, as shown in FIGS. 1A and 1B, and is preferably fabricated to comprise at least first and second layers 14a and 14b defining, respectively, the front side (FIG. 1A) and back side (FIG. 1B) of the unit 10. In the nonlimiting embodiment of FIGS. 1A and 1B, the substrate 12 has a generally planar (although flexible) form factor and generally has the shape of a triangular main body 26 and a short tab 28 extending centrally from an edge of the main body 26 (the bottom edge as viewed in FIGS. 1A and 1B) disposed generally opposite an apex of the main body 26 (the top corner as viewed in FIGS. 1A and 1B). Each of the three corners of the main body 26 are preferably, though not necessarily, rounded. Other shapes and forms for the substrate 12 are also possible. The sensor unit 10 is specifically configured for application to the orofacial musculature (head and neck) of a patient/user and is optimized for management of swallowing disorders. However other applications and uses related to at least speech, voice, orofacial myofunctional, and breathing disorders are also contemplated. As represented in FIG. 2, the triangular shape of the main body 26 is configured to be capable of generally following the contours of the patient/user's chin, with the apex of the main body 26 disposed under the apex of the chin and adjacent edges extending angularly outward from the apex generally along the edge of the mandible. The tab 28 extends posteriorly along the user's center line toward the throat. Thus, the generally triangular shape of the main body 26 makes it easier to place and align the sensor unit 10 in a preferred use location.

In one possible method, fabrication of the sensor unit 10 starts with fabrication of the two layers 14a and 14b of the substrate 12, including forming the layer 14a of non-stretchable polymeric material, as a nonlimiting example, a polyimide (PI) film or sheet, and forming the layer 14b of a metallic material, as a nonlimiting example, a copper (Cu) film or sheet. The nonlimiting embodiment of the sensor 10 unit shown in FIGS. 1A and 1B includes five sensor electrodes 16a, 16b, 16c, 16d, and 18. The materials of the layers 14a and 14b are preferably selected so that the substrate 12 is relatively flexible (capable of flexing out of the plane of the substrate 12) and yet relatively non-stretchable (limited strain in the plane of the substrate 12), enabling the sensor unit 10 as a whole to be relatively flexible yet non-stretchable. The sensor electrodes 16a-16d and 18 are represented in the form of pads, though other configurations are foreseeable. In one nonlimiting example, the layer 14a is a PI sheet that is approximately 70 micrometers thick, and the layer 14b is a Cu layer that is approximately 35 micrometers thick. Other materials and thicknesses for the layers 14a and 14b are foreseeable. As not limiting examples, alterative materials for the layer 14a include polyethylene naphthalate, polyester, and polyetherimide, and alternative materials for the layer 14b include conductive silver ink and carbon ink. To ensure adequate flexibility, maximum thicknesses for the layers 14a and 14b are believed to be 500 micrometers and 100 micrometers, respectively.

As represented in FIG. 1A, the sensor electrodes 16a-16d and 18 include four active electrodes 16a-16d and a single ground electrode 18, though it is foreseeable that fewer or more active electrodes 16a-16d and more ground electrodes 18 could be utilized. The sensor electrodes 16a-16d and 18 may be chemically etched in the first layer 14a at the front side of the substrate 12. In the nonlimiting embodiment shown in FIGS. 1A and 1B, the active electrodes 16a-16d are disposed between the ground electrode 18 and the tab 28 and are arranged to allow recording of muscle activity from bilateral muscle pairs of the orofacial musculature. The active electrodes 16a-16d may be configured as double differential electrodes. For example, the active electrodes 16a and 16c may be arranged and interconnected to form a first electrode pair, and the active electrodes 16b and 16d may be arranged and interconnected to form a second electrode pair. The first and second electrode pairs are disposed on opposite sides of a medial axis 32 of the main body 26 extending from the apex through a mid-point of the opposite bottom edge of the body 26, which in this case also extends through the mid-line of the tab 28. The first and second electrode pairs are also shown as being arranged symmetrically on opposite sides of the medial axis 32 of the main body 26. The active electrodes 16a-16d are arranged so that they will be near to and thereby able to record submental muscle activity bilaterally when in a suitable use position such as represented in FIG. 2. The active electrodes 16a-16d are preferably sized to be proportional in size to the muscles of interest. As a nonlimiting example, the interelectrode distance between the active electrodes 16a-16d may be approximately 1.5 cm from edge to edge so that the active electrodes 16a-16d are aligned with the submental muscle fibers when properly aligned in the use position as shown in FIG. 2, though other spacings may be more appropriate or used. In embodiments such as represented in FIGS. 1A, 1B, and 2, the inclusion of all the sensor components, particularly the active electrodes 16a-16d in combination with the ground electrode 18, within the same self-contained sensor unit 10 is believed to enable muscle activity signals obtained from bilateral muscle pairs to be more robust, reduces cost and complexity of the unit 10, and/or facilitates placement of the sensor unit 10.

The ground electrode 18 is arranged so as to come into direct contact with the middle line of the mandible (just below the mental protuberance) of a patient as represented by the nonlimiting position shown in FIG. 2. As best seen in FIGS. 1A, 1B, and 2, the ground electrode 18 is disposed generally near or adjacent the apex of the main body 26. In investigations leading to the invention, the arrangement of electrodes 16a-16d and 18 represented in FIGS. 1A, 1B, and 2 has been shown to improve signal quality by making the sensor unit 10 self-contained and eliminating the need for a separate ground electrode, which would otherwise add cost and complexity to the apparatus. Further, this arrangement of the ground electrode 18 provides a convenient point of reference for placement onto a patient's skin. This arrangement of the ground electrode 18 may help patients with consistent self-placement of the sensor in the submental area, which can be challenging. In preferred but nonlimiting embodiments, the electrodes 16a-16d and 18 that interface the skin are plated with a thin layer of tin (Sn) and then a thin layer of gold (Au) to ensure biocompatibility.

As more readily apparent in FIG. 1B, traces (collectively identified as traces 20, individually identified as traces 20a, 20b, 20c, 20d, and/or 20e) transfer electrical signals from respective electrodes 16a, 16b, 16c, 16d, and 18 to one or more other components of a biofeedback device, such as a wireless communications unit (not shown). The traces 20 are formed, for example by being chemically etched, on the back side of the unit 10 in the second layer 14b of the substrate 12. The traces 20 are connected to the respective electrodes 16a-16d and 18 through miniaturized vias 22. The traces 20 extend from the vias 22 to the tab 28 located at the bottom edge of the main body 26 opposite its apex. The tab 28 serves as an electrical connector adapted to be connected to wires 30, such as 26-gauge wires, by any suitable means, for example with solder. The wires 30 can connect the substrate 12 with a wireless data communication unit (not shown), which transmits data received from the electrodes 16a-16d for further processing, for example to a processor running custom-made specialized software. A via 22 is located directly behind each of the electrodes 16a-16d and 18. Each via 22 extends at least partly through the substrate 12 between and connecting one of the traces 20 with its respective electrode 16a-16d and 18. Arranging the sensor unit 10 as a double-sided component with the electrodes 16a-16d and 18 on one side (e.g., the front side) of the substrate 12 and the traces 20 on the opposite side (e.g., the back side) of the substrate 12 allows separation of the electrodes 16a-16d from the traces 20, which it is believed can eliminate opportunities for contamination of the electrical signal and increase durability of the device.

Reinforcing layers 24, such as thin layers of Cu, are disposed on opposite sides of the substrate 12 at two interior corners of the body 26 located on opposite sides of the tab 28, in the area where the bottom edge of the main body 26 intersects the tab 28. The reinforcing layers 24 inhibit tearing at the two interior corners adjacent the tab 28, which are high-stress points where tearing is more likely to occur, to increase the durability and life of the sensor unit 10.

The sensor unit 10 is not represented as including an integrated adhesive layer on the front side of the unit 10 for adhering the unit 10 and its electrodes 16a-16d and 18 to the skin of an individual, though it is foreseeable that an integrated adhesive layer could be incorporated into the unit 10. In the absence of an integrated adhesive layer, an adhesive may be applied to the front side of the unit 10 by the patient, technician, or other user immediately or shortly before pressing the front side of the unit 10 to the patient's skin. In a nonlimiting example, a conductive paste widely used in neuromonitoring procedures, such as Ten20® Conductive Paste available from Weaver and Company, may be manually applied prior to applying the sensor unit 10 to the skin. Optionally, a piece of skin safe adhesive tape may be used for adhesion to the skin.

As best seen in FIG. 2, the wearable sensor unit 10 is shaped and sized for being placed in an operative position on the submental area of a user/patient with the apex and the ground electrode 18 in a forward position aligned under the forward apex of the chin and the tab 28 extending rearward toward and generally aligned with the throat. However, the wearable sensor unit 10 can easily be re-configured and adapted to record electrophysiological muscle activity from any orofacial area.

FIGS. 3A and 3B depict an additional configuration of wearable sEMG sensor unit 100 in accordance with a further embodiment of this invention. In these figures, consistent reference numbers are used to identify the same or functionally equivalent elements. In FIGS. 3A and 3B, the sensor unit 100 is shown as comprising a body 26 having a shape roughly equivalent to an isosceles trapezoid. As with the unit of FIGS. 1A and 1B, the sensor unit 100 comprises a substrate 12 that is preferably thin, flexible, and non-stretchable, and has at least first and second layers 14a and 14b defining, respectively, the front side (FIG. 3B) and back side (FIG. 3A) of the unit 100. Each corner of the main body 26 is preferably, though not necessarily, rounded. The shape of the main body 26 is configured to be capable of generally following the contours of a patient/user's chin, with the shorter side of the isosceles trapezoid shape adapted to be disposed under the apex of the chin and adjacent edges (legs of the isosceles trapezoid shape) extending angularly outward from the shorter side generally along the edge of the mandible. This embodiment omits the ground electrode 18 of FIGS. 1A and 1B.

Sensor units 10 configured as represented in FIGS. 1A, 1B, and 2 were tested for adhesion, signal consistency, and technical durability after repeated uses. Twenty applications/uses of sensor units 10 on the submental area were examined on two individual subjects, providing a total of forty test applications. The technical integrity of the sensor units 10 was tested through repeated uses/applications in two young adult subjects. Each individual subject was trained to independently place the sEMG sensor unit 10 on the submental skin area and perform a standard set of swallows (of liquids and solids) and swallow maneuvers (tongue presses, hard swallows) while the signals from the sensor units 10 were recorded on a software platform connected to a commercial wireless unit (Bioradio). Each sensor unit 10 was applied a total of twenty times on each individual subject. If signal degradation was noted before the end of the twentieth use, signal data collection ceased, but placement and removal procedures continued to examine skin adhesion and durability for twenty applications.

As discussed in more detail below, regarding adhesion, additional gel application was needed three times throughout the forty uses. Regarding signal consistency, signal quality remained strong through the 16^{th} and 19^{th} uses of the first and second individual, respectively, which is double the lifetime of other ultrathin wearable swallowing sEMG sensor units. Signal to noise (SNR) ratio ranged from 18 to 24 across all sessions, which is equal or higher to that reported in prior similar work. No tearing of either sensor unit 10 was observed at any time.

Data collection procedures followed a standard protocol as described, for example, in Kantarcigil and elsewhere. In summary, the data collection procedures were completed by a trained research assistant (RA), who first cleaned the submental skin of the subjects with alcohol wipes to reduce skin—electrode impedance and further applied tape to the skin to remove any additional extraneous tissue/dirt. Then, the subjects were trained, via a step-by-step written manual, to adhere the wearable sensor unit 10 to their submental area, connect it to the wireless unit, and use the software for data acquisition. For placement, the ground electrode 18 (that specifically aligns to the lower part of the mandible's mental protuberance) was used to guide correct placement. The RA was available to help with this procedure, but for the most part the written instructions were adequate to allow independent data recording by the subjects. As it has been described in Kantarcigil, the sEMG signal was pre-amplified with a gain of 1000 and fourth-order Butterworth bandpass filtered with low and high cutoff frequencies of 20 and 500 Hz. A 60-Hz notch filter was used to reduce any powerline interference, and a sampling rate of 1000 samples per second was used.

Before each data collection, subjects were asked to sit as still as possible and breathe normally for 30 seconds to obtain a baseline resting sEMG amplitude, and then the subjects completed a criterion-reference task comprising maximum voluntary contraction of the submental muscles. This was a maximum anterior tongue press using the Iowa Oral Performance Instrument, where an air-filled bulb is placed on the anterior tongue and subjects are asked to push the bulb against the roof of their mouth as hard as possible. Three maximum effort anterior lingual pressure values (in kilopascals) were recorded, and the average of these trials was used to normalize the sEMG signal during data analysis.

Outcome variables examined included: adhesion, signal consistency, and technical durability across uses.

Adhesion to the skin was examined by noting the times adhesion was reduced and additional conductive gel application was needed across the twenty applications. The RA was in charge of recording these observations. Also, the RA visually inspected the submental area and took photos before, during removal, and immediately after the removal of the unit 10 for each session.

To examine consistency of signal quality across sessions for each individual, the signal to noise ratio (SNR) was examined across uses. EMG signals obtained from the left and right submental muscles were analyzed separately. A custom-written MATLAB script (MATLAB Inc.) with critical pre- and postprocessing steps including filtering, demeaning, full-wave rectification, and smoothing was developed. Before post-processing, the raw sEMG signal was visually inspected for motion artifacts, and any artifact that occurred during the rest periods was removed. Signal to Noise ratio was calculated using the following equation:

$$SNR = 20 {log}_{10} \frac{Signal}{Noise} . \qquad Equation 1$$

Technical durability was examined by visually inspecting the wearable sensor unit 10 for tears or destruction throughout its components and by taking photographs of the sensor unit 10 before and after each application/use.

### Results

### Adhesion & Visual Inspection

Thirty seven out of the forty total applications/uses were completed without any adhesion issues. Additional conductive gel application was needed three times throughout the forty sessions. Visual inspection revealed no issues with skin appearance.

### Consistency of Signal Quality and Technical Durability

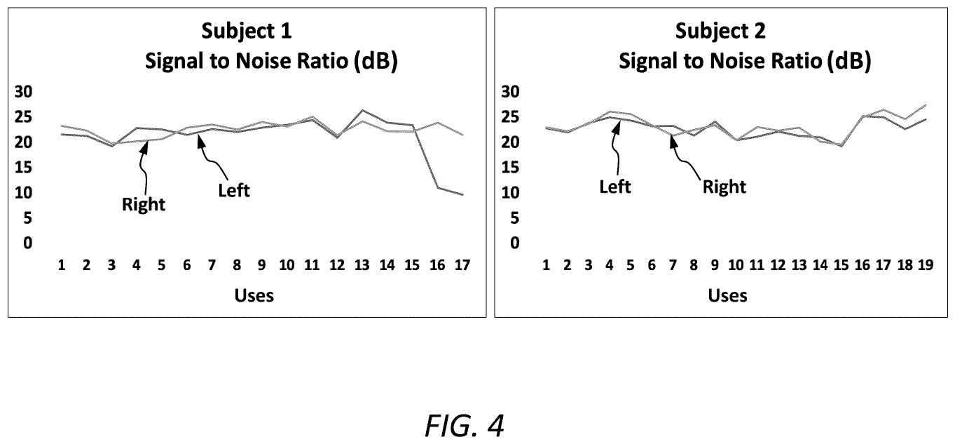

As seen in FIG. 4, signal quality remained strong (i.e., SNR >18) for 16 and 19 uses for the first and second individual, respectively. Signal to noise ratio ranged from 18 to 24 across all sessions. In addition, no tearing was observed at any time on any of the components of the wearable sensor units 10.

Technical durability is an important feature for clinical adoption as it relates to re-usability and cost and is important for future clinical use. The data show that the sensor unit 10 provided good technical durability. The wearable sEMG sensor units 10 exhibited high technical and durability aspects of its performance. The sensor units 10 had excellent technical durability (twenty uses) without any tearing observed across any of the components of the units 10. Without being bound by theory, it is believed that design features that led to this improvement may include one or more of the thicker and non-stretchable substrate 12 and the inclusion of the reinforcing layers 24 at high-stress regions of the main body 26 of the substrate 12.

In addition to being durable from a materials standpoint, signal quality remained high (SNR >18) across 16 and 19 uses for each individual, respectively. Without being bound by theory, it is believed that this may be due to factors related to electrode type and placement. For example, double differential electrodes 16a-16d were incorporated to record submental muscle activity bilaterally that were proportional in size to the muscles of interest. Also, a skin preparation protocol for electrode adherence to the skin was used, and very good skin adherence for the majority of trials (37/40) was observed. Further, the sensor unit 10 included the ground electrode 18 within the same substrate/interface, the first layer 14a, which enabled easier and more consistent placement of the unit 10 across subjects and trials. Although signal degradation appeared to initiate on the 17th and 20th trial of each individual test subject, signal quality remained excellent until these time points.

The wearable sEMG sensor units 10 exhibited good adhesion, excellent technical durability, and reliable signal quality across multiple uses. It is believed that, among other possible uses and advantages, the sensor units 10 may provide an affordable and reliable sEMG biofeedback solution for the treatment and tele-treatment of patients with dysphagia.

As previously noted above, though the foregoing detailed description describes certain aspects of one or more particular embodiments of the invention, alternatives could be adopted by one skilled in the art. For example, the sensor unit 10 and its components could differ in appearance and construction from the embodiments described herein and shown in the drawings, functions of certain components of the sensor unit 10 could be performed by components of different construction but capable of a similar (though not necessarily equivalent) function, and various materials could be used in the fabrication of the sensor unit 10 and/or its components. As such, and again as was previously noted, it should be understood that the invention is not necessarily limited to any particular embodiment described herein or illustrated in the drawings.

Claims:

1. A wearable surface electromyography (sEMG) sensor unit comprising:
a flexible non-stretchable substrate comprising a first layer defining a first side of the substrate and a second layer defining a second side of the substrate;
a plurality of sensor electrodes disposed on the first side, the plurality of sensor electrodes including at least a first pair of active electrodes and a ground electrode;
a plurality of traces disposed on the second side; and
vias connecting the traces to the sensor electrodes.

2. The wearable sEMG sensor unit of claim 1, wherein the first layer comprises a non-stretchable polymeric sheet.

3. The wearable sEMG sensor unit of claim 1, wherein the non-stretchable polymeric sheet is a polyimide material.

4. The wearable sEMG sensor unit of claim 1, wherein the second layer is a metallic layer.

5. The wearable sEMG sensor unit of claim 4, wherein the metallic layer is copper.

6. The wearable sEMG sensor unit of claim 1, wherein the plurality of sensor electrodes further comprises a second pair of active electrodes on the first side, the first and second electrode pairs being symmetrically arranged on opposite sides of a medial axis of the substrate.

7. The wearable sEMG sensor unit of claim 1, wherein the substrate comprises a main body having a generally triangular shape.

8. The wearable sEMG sensor unit of claim 7, wherein the substrate comprises a tab extending from an edge of the body opposite an apex of the main body.

9. The wearable sEMG sensor unit of claim 8, wherein the ground electrode is disposed adjacent the apex, and wherein the first pair of active electrodes is disposed between the ground electrode and the edge of the main body.

10. The wearable sEMG sensor unit of claim 1, further comprising a reinforcing layer disposed on the substrate, the reinforcing layer being located at a high stress area of the substrate to reduce tearing of the substrate.

11. The wearable sEMG sensor unit of claim 10, wherein the reinforcing layer is disposed at an interior corner defined by a tab of the substrate and a main body of the substrate.

11. The wearable sEMG sensor unit of claim 1, wherein the first pair of active electrodes are arranged to enable sensing of muscle activity from bilateral muscle pairs of the orofacial musculature.

12. The wearable sEMG sensor unit of claim 1, wherein the sEMG sensor unit is configured for application to the orofacial musculature of a patient and is configured to optimize management of swallowing disorders.

13. The wearable sEMG sensor unit of claim 1, wherein the wearable sEMG sensor unit does not include an integrated adhesive for adhering to a patient's skin.

14. The wearable sEMG sensor unit of claim 1, wherein the first pair of active electrodes comprises differential electrodes.

15. The wearable sEMG sensor unit of claim 1, wherein each of the first pair of active electrodes is proportional in size to a muscle of the orofacial musculature.

16. A method of obtaining surface electromyography (sEMG) biofeedback for the treatment of a patient with dysphagia, the method comprising:
placing the wearable sEMG sensor unit of claim 1 in a use position on the submental area of the patient such that each active electrode of the first pair of active electrodes is located near a submental muscle of interest; and
obtaining signals from the first pair of active electrodes indicative of activity of the submental muscle of interest.

17. The method of claim 16, wherein the step of placing comprises locating the ground electrode in direct contact with a middle line of the patient's mandible just below the mental protuberance.

18. The method of any of claim 16, wherein the placing of the wearable sEMG sensor unit comprises adhering the sEMG sensor unit in the use position.

19. A method of fabricating the wearable sEMG sensor unit of claim 1, comprising:
overlaying a non-stretchable polymeric layer with a metallic layer;
forming the sensor electrodes on the first side;
forming the traces on the second side; and
connecting each of the sensor electrodes to a respective one of the traces with a respective one of the vias.
